(19) United States
(12) Patent Application Publication
(10) Pub. No.: US 2025/0381053 A1
(43) Pub. Date: Dec. 18, 2025

(54) SCOLIOSIS CORRECTION GARMENT

(71) Applicants: The Hong Kong Polytechnic University, Hong Kong (CN); The Chinese University of Hong Kong, Hong Kong (CN); The University of Hong Kong-Shenzhen Hospital, Shenzhen (CN)

(72) Inventors: Yiu-Wan Joanne Yip, Hong Kong (CN); Kai-Yu Raymond Tong, Hong Kong (CN); Man-Chee Kenneth Cheung, Hong Kong (CN); Qiwen Lei, Hong Kong (CN); Jinyi Ma, Hong Kong (CN); Zhongping Ye, Hong Kong (CN); Shuk Fan Tong, Hong Kong (CN)

(73) Assignees: The Hong Kong Polytechnic University, Hong Kong (CN); The Chinese University of Hong Kong, Hong Kong (CN); The University of Hong Kong-Shenzhen Hospital, Shenzhen (CN)

(21) Appl. No.: 19/015,896

(22) Filed: Jan. 10, 2025

(30) Foreign Application Priority Data
Jun. 14, 2024 (CN) 202410773009X

(51) Int. Cl. A61F 5/02; A61F 5/34

(52) U.S. Cl. CPC A61F 5/024; A61F 5/34

(57) ABSTRACT

The present disclosure provides a scoliosis correction garment. The garment includes a garment body provided with a fixing portion and a filling pad detachably fixed to the garment body through the fixing portion, and a corrective pressure is adjusted by controlling gas filling in the filling pad, so as to apply a corrective pressure to a scoliosis position of a user.

### CROSS REFERENCE

The present application is based on and claims priority to Chinese Patent Application No. 202410773009X, filed on Jun. 14, 2024, the entire contents of which are incorporated herein by reference.

### TECHNICAL FIELD

The present disclosure relates to the field of health equipment, and in particular to a scoliosis correction garment.

### BACKGROUND

Patients with adolescent idiopathic scoliosis are offered different treatment methods according to their own spinal situation. Treatment decisions of the orthotist are basically based on whether the spinal curvature progresses as the patient matures. The choice of treatment basically depends on the severity of the curvature of the spine, its location and risk of progression, and the Risser sign of the patient. Among several non-surgical treatments, wearing a brace is the most widely used method to manage this condition. Braces can maintain the patient's body balance and prevent the progression of the spinal curvature before the patient reaches skeletal maturity. To prevent spinal deformity through the use of a brace until a patient reaches skeletal maturity is the main goal of bracing treatment.

It should be noted that the information disclosed in the Background section above is only for enhancing the understanding of the background of the present disclosure, and thus may include information that does not constitute prior art known to those of ordinary skill in the art.

### SUMMARY

According to an aspect of the present disclosure, there is provided a scoliosis correction garment, including: a garment body, provided with a fixing portion; and a filling pad, detachably fixed to the garment body through the fixing portion to apply a corrective pressure to a scoliosis position of a user, wherein the corrective pressure is adjusted by controlling gas filling in the filling pad.

It should be noted that the above general description and the following detailed description are merely exemplary and explanatory and should not be construed as limiting of the disclosure.

### BRIEF DESCRIPTION OF THE DRAWINGS

The above and other objectives, features and advantages of the present disclosure will become more apparent by describing in detail example embodiments thereof with reference to the attached drawings.

FIG. 1A shows a front schematic diagram of a scoliosis correction garment according to an embodiment of the present disclosure.

FIG. 1B shows a back schematic diagram of a scoliosis correction garment according to FIG. 1A.

FIG. 1C shows a side schematic diagram of a scoliosis correction garment according to FIG. 1A.

FIG. 2 shows a schematic diagram of a filling pad and a corresponding pocket according to an embodiment of the present disclosure.

FIG. 3 shows a schematic diagram of a connection of a pocket with a garment body through several first fixing positions according to an embodiment of the present disclosure.

FIG. 4 shows an example connection manner in which a pocket is connected with a garment body through several first fixing positions.

FIG. 5 shows another example connection manner in which a pocket is connected with a garment body through several first fixing positions.

FIG. 6 shows a schematic diagram of a connection of a pocket with a second fixing portion of a garment body through a third fixing position according to an embodiment of the present disclosure.

FIG. 7A shows a front schematic diagram of a scoliosis correction garment having a filling pad with a different shape from that of FIG. 1A according to an embodiment of the present disclosure.

FIG. 7B shows a back schematic diagram of a scoliosis correction garment according to FIG. 7A.

FIG. 8 shows an X-ray perspective view of a C-shaped scoliosis according to an embodiment of the present disclosure.

FIG. 9 shows an X-ray perspective view of an S-shaped scoliosis according to an embodiment of the present disclosure.

FIG. 10A shows a schematic diagram of three filling pads of different shapes according to an embodiment of the present disclosure.

FIG. 10B shows a schematic diagram of corresponding three pockets of different shapes from an inner side perspective according to FIG. 10A.

FIG. 10C shows a schematic diagram of three filling pads of different shapes placed in corresponding pockets according to FIGS. 10A and 10B.

FIG. 10D shows a schematic diagram of three pockets of different shapes fixed at positions of corresponding first fixing positions according to FIG. 10B.

FIG. 11A shows a schematic diagram of a pneumatic circuit according to an embodiment of the present disclosure.

FIG. 11B shows a schematic diagram of an example physical connection of the pneumatic circuit diagram in FIG. 11A.

FIG. 12 shows a schematic diagram of another pneumatic circuit according to an embodiment of the present disclosure.

FIG. 13 shows a flowchart schematic diagram of controlling gas in a filling pad through a pneumatic circuit according to an embodiment of the present disclosure.

FIG. 14 shows a schematic diagram of a shoulder fixing belt according to an embodiment of the present disclosure.

FIG. 15 shows a schematic diagram of a pelvic fixing belt according to an embodiment of the present disclosure.

FIG. 16A shows a front schematic diagram of a wearing manner of a shoulder fixing belt and a pelvic fixing belt according to an embodiment of the present disclosure.

FIG. 16B shows a back schematic diagram of a wearing manner of a shoulder fixing belt and a pelvic fixing belt according to an embodiment of the present disclosure.

FIG. 16C shows a side schematic diagram of a wearing manner of a shoulder fixing belt and a pelvic fixing belt according to an embodiment of the present disclosure.

FIG. 17 shows a schematic diagram of a user actually wearing a clothing including a shoulder fixing belt and a pelvic fixing belt according to FIGS. 16A to 16C.

FIG. 18A shows a front schematic diagram of another wearing manner of a shoulder fixing belt and a pelvic fixing belt according to an embodiment of the present disclosure.

FIG. 18B shows a back schematic diagram of another wearing manner of a shoulder fixing belt and a pelvic fixing belt according to an embodiment of the present disclosure.

FIG. 18C shows a side schematic diagram of another wearing manner of a shoulder fixing belt and a pelvic fixing belt according to an embodiment of the present disclosure.

FIG. 19 shows a schematic diagram of a user actually wearing a clothing including a shoulder fixing belt and a pelvic fixing belt according to FIGS. 18A to 18C.

FIG. 20 shows a schematic structural diagram of an electronic device according to an embodiment of the present disclosure.

### DETAILED DESCRIPTION

Example embodiments will now be described more fully with reference to the accompanying drawings. However, the example embodiments can be implemented in a variety of forms and should not be construed as being limited to examples set forth herein; rather, these embodiments are provided so that the present disclosure will be more complete and comprehensive so as to convey the idea of the example embodiments to those skilled in this art. The drawings are merely schematic representations of the present disclosure and are not necessarily drawn to scale. The same reference numerals in the drawings denote the same or similar parts, and the repeated description thereof will be omitted.

In addition, the described features, structures or characteristics may be combined in any suitable manner in one or more embodiments. In the following description, many specific details are provided to give a full understanding of the embodiments of the present disclosure. However, those skilled in the art will appreciate that the technical solution of the present disclosure may be practiced without one or more of the specific details, or other methods, devices, steps and the like may be employed. In other instances, well-known structures, methods, devices, implementations or operations are not shown or described in detail to avoid obscuring various aspects of the present disclosure.

In addition, the terms “first”, “second”, etc. are used for descriptive purposes only and should not be understood as indicating or implying relative importance or implicitly indicating the number of the indicated technical features. Therefore, the features defined with “first” or “second” may explicitly or implicitly include one or more of the features. In the description of the present disclosure, the meaning of “plurality” is at least two, such as two, three, etc., unless otherwise clearly and specifically defined. The symbol “/” generally indicates that objects associated with each other are in an “or” relationship.

In the present disclosure, unless otherwise clearly specified and limited, the terms such as “connection” should be understood in a broad sense, for example, it can be an electrical connection or mutual communication; it can be direct connection or indirect connection through an intermediate medium. For those skilled in the art, the specific meanings of the above terms in this disclosure can be understood according to specific contexts.

Two types of braces are designed in the related arts, which are respectively rigid braces and soft braces. Patient compliance plays an important role in a decision of which type of brace to use in treatment. The primary means of monitoring compliance with bracing treatment include asking the patient to complete a user logbook, evaluating the conditions of the skin, and checking the amount of spinal correction with the donned brace. These methods are based on the patient ensuring a certain wearing time. Many researches have been done to increase the compliance rate of bracing treatment for adolescent idiopathic scoliosis. For example, soft braces have been designed and developed in the related arts that ensure wear comfort and do not limit body movement. However, a correction success rate of the soft brace is still lower than that of a certain rigid orthosis. More research work needs to be done to increase the success rate of soft braces in controlling spinal deformities.

The purpose for offering non-surgical treatment (such as wearing the brace) for scoliosis patients is to offer a conservative solution to treat their condition and encourage a higher degree of compliance by avoiding interference with their daily life activities. Therefore, nighttime braces have been developed to solve this issue, which are intended to be worn only while the patient is sleeping. The design principle is to overcorrect the major scoliotic curve so as to reduce the brace wear time to a minimum of 8 hours each night during sleeping hours.

In the relevant researches, treatment outcomes of nighttime braces and full-day braces are compared, which show that a treatment success rate with the nighttime braces is comparable to that with the full-day braces, which are worn almost all day. Therefore, the nighttime braces can be shown to be an effective means of treating patients with adolescent idiopathic scoliosis. However, even though there is a shorter usage time, wearing braces at night runs the risk of overcorrection and skin issues. For instance, after the patient uses the nighttime brace, bursas, rashes, and skin reddening have been observed. In addition, it has been noted that some nighttime braces might result in an increase in compensatory curves due to the possibility that the uneven loads on the vertebrae will result in unintended corrective forces. Overcorrecting spinal curvatures can be a significant and permanent issue with negative repercussions.

While nighttime braces have been studied for decades, and several different nighttime braces can be found in the market, their effectiveness is still worth exploring and modifications can be made as bracing during the night continues to show the highest degree of compliance.

Therefore, the present disclosure provides a scoliosis correction garment that can be worn at night, which is designed with a garment body provided with a fixing portion and a filling pad that is detachably fixed to the garment body through the fixing portion. A corrective pressure is adjusted by controlling gas filling in the filling pad to apply the corrective pressure to a scoliosis position of a user, thereby improving the effectiveness of correcting scoliosis.

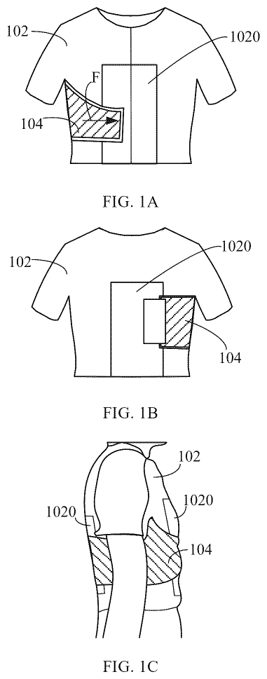

FIG. 1A shows a front schematic diagram of a scoliosis correction garment according to an embodiment of the present disclosure. The garment as shown in FIG. 1 may include a garment body 102 and a filling pad 104. The garment body 102 may be provided with a fixing portion 1020, and the filling pad 104 may be detachably fixed to the garment body 102 through the fixing portion 1020 to apply a corrective pressure to a scoliosis position of a user. The corrective pressure is adjusted by controlling the gas filling in the filling pad.

In some embodiments, the garment body 102 may be a top, such as a short-sleeved, medium-sleeved, or long-sleeved top, etc. FIGS. 1A to 1C are illustrated by taking the short-sleeved top as an example, but the present disclosure is not limited thereto.

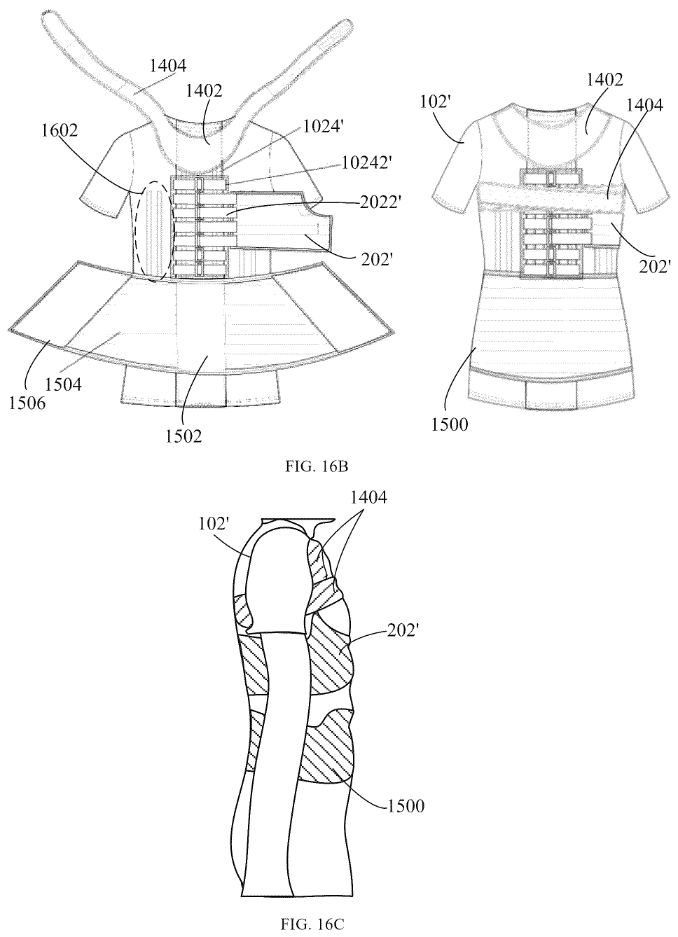

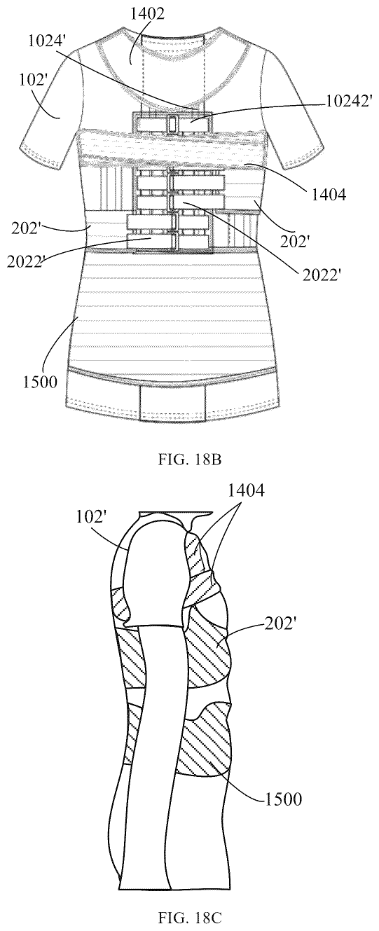

In some embodiments, the garment body may also be a bodysuit, for example, a short-sleeved, medium-sleeved, or long-sleeved bodysuit, etc. FIGS. 16A to 16C and FIGS. 18A to 18C show an example embodiment in which the garment body (102′) is the bodysuit, and the short-sleeved bodysuit is taken as an example for description, but the present disclosure is not limited thereto.

In some embodiments, the garment body 102 is made of at least one of: 100% cotton, cotton blended, or synthetic fiber blended. The cotton blended may be, for example, cotton spandex, and the synthetic fiber blends may be, for example, nylon spandex or polyester spandex, etc. The garment provided in embodiments of the present disclosure may be used as (close fitting) undergarment, middle clothing, outer clothing, etc. Different types of clothing can use different materials as needed, which is not limited by the present disclosure.

Taking the garment body 102 as the short-sleeved undergarment as an example, its main component may be made of 100% cotton, or cotton spandex, or nylon spandex, or polyester spandex to provide a comfortable and skin-friendly feel.

In some embodiments, the garment body 102 may be designed in a variety of sizes, such as the common XS size, S size, M size, L size, XL size, etc., for selection by wearers of different body shapes.

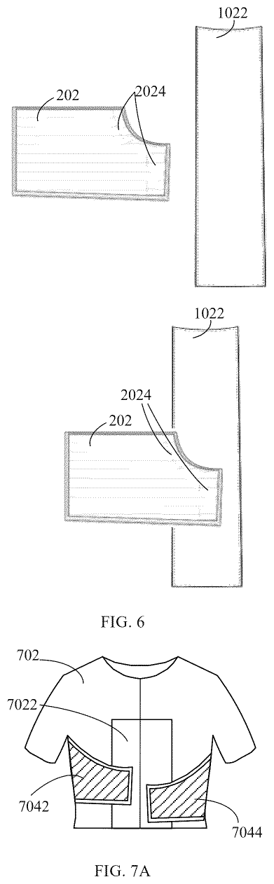

In some embodiments, the fixing portion 1020 (referred to as a second fixing portion 1022 in FIG. 6) may be provided in the middle of the front of the garment body 102, as shown in FIG. 1A, and/or the fixing portion 1020 (referred to as a first fixing portion 1024 in FIG. 3) may be provided in the middle of the back of the garment body 102, as shown in FIG. 1B (when only one of the front and back fixing portions 1020 is provided, the filling pad 104 may be provided with another fixing structure on the inner side of the other end, which is not limited by the present disclosure). When the filling pad 104 is fixed to the garment body 102, it may be fixed more evenly on the front and back of the garment body 102, so that a pressure applied to the vertebra of the user (for example, a patient) when wearing the garment is close to a plane produced by the curvature of the vertebra.

In some embodiments, the fixing portion 1020 may be a separate cloth from the garment body 102 and sewn onto the garment body 102; and the fixing portion 1020 may also be a portion of the cloth that forms the garment body 102.

In some embodiments, a shape of the filling pad 104 may be designed according to the user's body contour. For example, the patient's contour is scanned, and the shape of the filling pad 104 is designed according to the scanned image (such as FIGS. 8 and 9). The filling pad 104 may not be inflated before wearing, and after the patient wears the garment body 102, the filling pad 104 is fixed according to the scanned image so that its center is close to, in a horizontal direction, be aligned with the largest curvature position of the spine curve (for example, a direction in which the force is applied corresponding to F in FIG. 1A points to the largest curvature position of the patient's spine curve), and then inflated to an appropriate pressure, which makes the garment better fit the wearer's body shape and ensures that the filling pad can better adapt to the patient's body. Due to the increase in contact area, the filling pad can provide the same amount of corrective force as a traditional brace, but with less pressure applied to the patient.

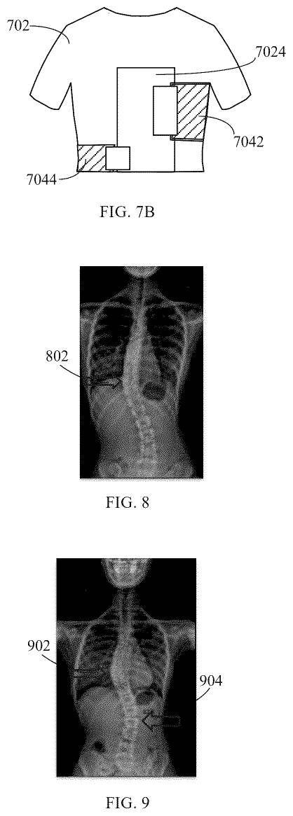

In some embodiments, the filling pad may include at least two filling pads of different shapes, so that the filling pads of corresponding shapes are fixed to corresponding positions of the fixing positions according to a spinal curvature position of the user, and the corrective pressure is applied to the user wearing the correction garment. FIGS. 7A and 7B show clothing with filling pads of shapes different from those of FIGS. 1A, 1B, and 1C. FIGS. 8 and 9 respectively show two corresponding scoliosis curves.

In some embodiments, the filling pad 104 may be directly connected to the fixing portion on the garment body 102, for example, detachably fixed by means of a magic tape, a buckle, etc.

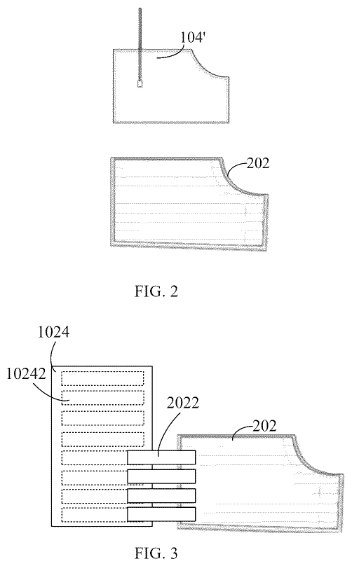

In other embodiments, the filling pad 104 may be placed in a pocket and fixed to the fixing portion of the garment body 102 through the pocket. For specific implementations, reference may be made to FIGS. 2 to 6.

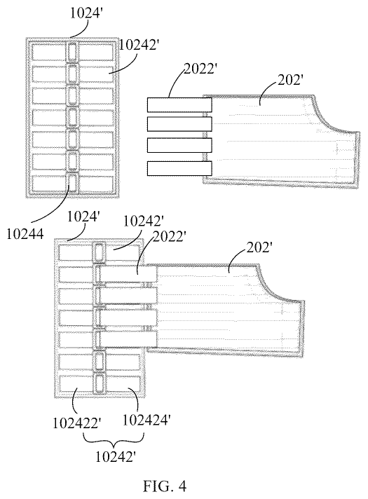

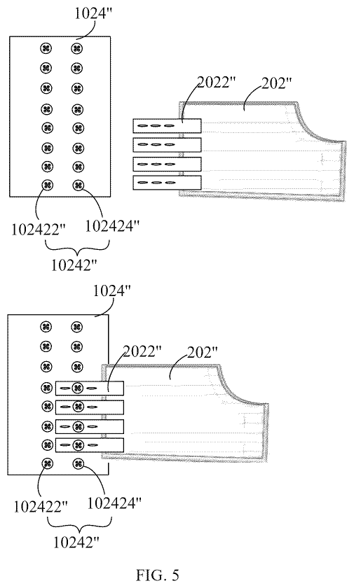

In some embodiments, the fixing portion 1020 may be implemented in a variety of ways. For example, FIG. 3 exemplarily shows a fixing manner in which the fixing portion is provided with a plurality of fixing positions, FIG. 4 and FIG. 5 exemplarily show fixing portions with two different fixing structures, respectively, and FIG. 6 exemplarily shows another fixing manner of a fixing portion. The front and back of the garment body may adopt different or the same implementations as in FIG. 3 to FIG. 6, which is not limited by the present disclosure. (Although an example in which the filling pad is placed in the pocket is given for illustration in FIGS. 3 to 6, the fixing manner may also be applied to the case where the filling pad is directly fixedly connected to the garment body).

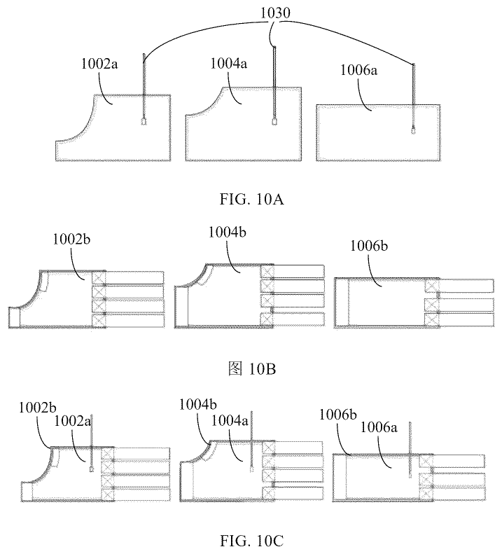

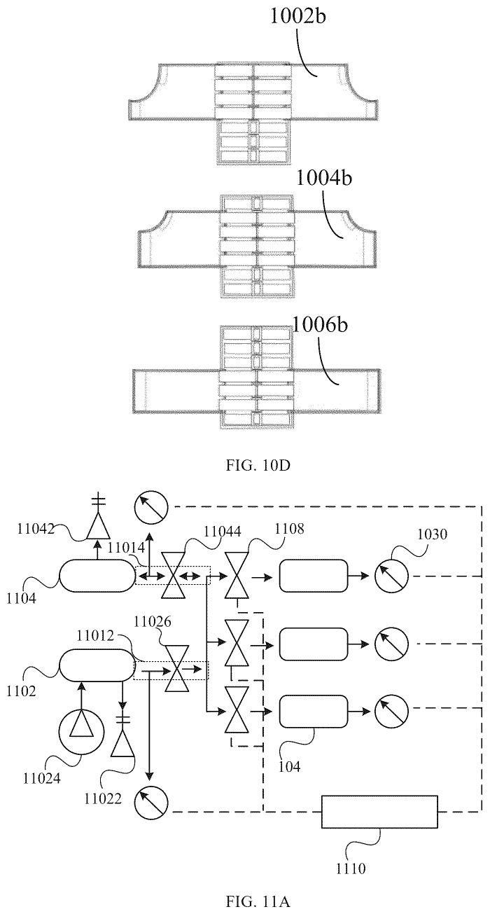

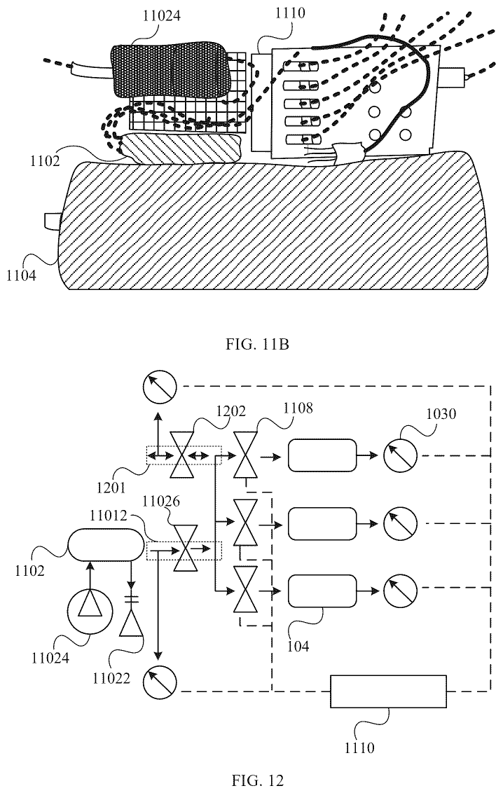

In some embodiments, the corrective pressure may be adjusted by controlling the gas filling in the filling pad 104 through a pneumatic circuit. For example, the pneumatic circuit may include an inflation line, a supply airbag, and an inflation valve. A first end of the inflation line is connected to the filling pad, a second end of the inflation line is connected to the supply airbag, an initial pressure of the supply airbag is higher than a first threshold, and the inflation valve is provided on the inflation line, so that when a measured value of a pressure sensor (such as 1030 in FIG. 10A) arranged on the filling pad is less than a second threshold, the inflation valve is controlled to open to inflate the filling pad 104 to a preset standard pressure. For some example implementations, reference may be made to FIGS. 11A to 13.

According to the scoliosis correction garment provided by the embodiments of the present disclosure, the garment body provided with the fixing portion and the filling pad detachably fixed to the garment body through the fixing portion are designed, and the corrective pressure is adjusted by controlling the gas filling in the filling pad to apply the corrective pressure to the scoliosis position of the user. The fixing portion is designed so that the user can find the appropriate correction position, which effectively improves the effectiveness of scoliosis correction; and the filling pad is used to increase the contact area between the force-applying portion and the body, so that the pressure applied to other parts of the user's body is smaller while providing the same corrective force as the traditional brace, which effectively improves the user's compliance.

FIG. 1B shows a back schematic diagram of a scoliosis correction garment according to FIG. 1A. FIG. 1C shows a side schematic diagram of a scoliosis correction garment according to FIG. 1A, and FIG. 1C shows a right view of the body. As shown in FIGS. 1B and 1C, the filling pad 104 wraps the (right side) body and is fixed to the front and back of the garment body 102 through the fixing portions 1020, respectively.

FIG. 2 shows a schematic diagram of a filling pad and a corresponding pocket according to an embodiment of the present disclosure. The filling pad 104 in FIGS. 1A to 1C may be placed in a pocket 202 and fixed to the fixing portion 1020 of the garment body 102 through the pocket. As shown in FIG. 2, the filling pad is denoted as 104′ in FIG. 2, and the filling pad described in FIG. 2 may be the same as or different from the filling pad in FIGS. 1A to 1C.

In some embodiments, the pocket may include a flexible material and is configured to accommodate the filling pad, and the filling pad is fixed to the fixing portion through the pocket.

For example, the pocket may be formed by sewing a woven elastic band and an elastic and light-transmitting fabric so as to monitor a condition of the filling pad therein. The elastic and light-transmitting fabric may be, for example, a bidirectional woven fabric or an elastic mesh. The woven elastic band is used to fix and connect the elastic and light-transmitting fabric and detachably connect the fixing portion 1020. The woven elastic band may be, for example, implemented as a second fixing position 2022 in FIG. 3. The woven elastic band should have good air permeability and moisture discharge to maintain good thermal physiology and ergonomics and wearing comfort when the garment is worn at night.

In some embodiments, an edge of the pocket may be covered by a v-fold elastic band.

In some embodiments, the pocket and the fixing portion may be designed with corresponding fixing positions, so that the user can adjust the fixing position of the pocket more accurately. For specific implementations, reference may be made to FIGS. 3 to 5.

According to the pocket provided in the embodiments of the present disclosure, the filling pad may be placed therein and then fixed on the garment body, so that a material of the filling pad can focus on airtightness, a material of the pocket can focus on easy fixation, and the design of the shape of the filling pad and replacement of the filling pad due to wear, etc. are more flexible, which effectively improves the flexibility of the design of the scoliosis correction garment.

FIG. 3 shows a schematic diagram of a connection of a pocket with a garment body through several first fixing positions according to an embodiment of the present disclosure. FIG. 4 and FIG. 5 show two implementations in which connection is realized.

In FIG. 3, an example in which the fixing portion 1020 includes a first fixing portion 1024 disposed at the back of the garment body 102 is taken for illustration.

As shown in FIG. 3, the first fixing portion 1024 is provided with a plurality of first fixing positions 10242 (for example, 6, 7, 8, etc. first fixing positions), the pocket 202 is provided with at least one second fixing position 2022 (for example, 3, 4, etc. second fixing positions). The at least one second fixing position 2022 can be selectively fixed to a corresponding number of first fixing positions 10242 among the plurality of first fixing positions 10242, so that the pocket 202 is fixed to a selectable position of the fixing portion.

According to the plurality of fixing positions provided in the embodiments of the present disclosure, the pocket can be fixed to the selectable position of the fixing portion by selecting the first fixing position to which the at least one second fixing position on the pocket is fixed, so that the user can adjust the fixing position of the pocket more accurately, thereby improving the effectiveness of correcting scoliosis.

FIG. 4 shows an example connection manner in which a pocket is connected with a garment body through several first fixing positions. As shown in FIG. 4, the first fixing position 10242′ may include a connection buckle 10244 fixed to the first fixing portion 1024′, and the connection buckle 10244 may be, for example, sewn on the first fixing portion 1024′; and the second fixing position 2022′ may include a hook and loop magic tape or Velcro, and the hook and loop magic tape may pass through the connection buckle 10244 and bond itself.

The connection buckles 10244 can divide the plurality of first fixing positions 10242′ into two columns 102422′ and 102424′ along a longitudinal direction of the garment body, and each column of first fixing positions is used to fix second fixing positions 2022′ on the same side. For example, FIG. 10D illustrates a case where two sides of connection buckles respectively fix second fixing positions on their respective sides.

The first fixing positions 10242′ may also include a plurality of woven elastic bands fixed to the first fixing portion, the plurality of woven elastic bands are arranged in the longitudinal direction of the garment body and extend in a transverse direction of the garment body, the connection buckles are sewn on the first fixing portion, and the woven elastic bands pass through the corresponding connection buckles. Through the design of the woven elastic band, on the one hand, the connection buckles can be ensured to be in a horizontal direction to ensure a direction of a tension applied by the pocket or the filling pad fixed thereon, and on the other hand, the elasticity of the garment body can be increased, the tension increased by fixing the pocket or the filling pad thereon can be reduced, and the wearing comfort of the garment can be improved.

FIG. 5 shows another example connection manner in which a pocket is connected with a garment body through several first fixing positions. As shown in FIG. 5, the first fixing positions 10242′ may include buttons 10242″ fixed to the first fixing portion 1024″, and the second fixing positions 2022″ provided on the pocket 202″ may include woven elastic bands provided with buttonholes. The pocket 202″ may be fixed at a desired position by passing some buttons 10242″ on the first fixing portion 1024″ through the buttonholes on the woven elastic bands of the second fixing positions 2022″.

FIG. 6 shows a schematic diagram of a connection of a pocket with a second fixing portion of a garment body through a third fixing position according to an embodiment of the present disclosure. In FIG. 6, an example in which the fixing portion 1020 includes a second fixing portion 1022 disposed on the front of the garment body 102 is taken for illustration.

As shown in FIG. 6, the pocket 202 may be provided with at least one third fixing position 2024, and the at least one third fixing position 2024 is fixed to the second fixing portion 1022.

In some embodiments, the third fixing portion 2024 may include a hook-surface magic tape or Velcro, and the second fixing portion 1022 may include a loop-surface magic tape or Velcro.

According to the garment provided by the embodiments of the present disclosure, the separate pocket connected with the woven elastic bands is used to accommodate the filling pad, and the hook and loop magic tape on the woven elastic band is connected to a specified position of the fixing portion in the middle of the back of the garment body, and the magic tape on the pocket made of elastic and light-transmitting fabric material is connected to the fixing portion in the middle of the front of the garment body, so that the pocket accommodating the filling pad wraps around the body and applies the corrective force to the wearer's spine, which can effectively control the further increase of the spinal curvature.

FIG. 7A shows a front schematic diagram of a scoliosis correction garment having a filling pad with a different shape from that of FIG. 1A according to an embodiment of the present disclosure, and FIG. 7B shows a back schematic diagram of a scoliosis correction garment according to FIG. 7A. As shown in FIGS. 7A and 7B, a second fixing portion 7022 is provided in the middle of the front of the garment body 702, and a first fixing portion 7024 is provided in the middle of the back of the garment body 702. An end of the filling pad 7042 is detachably fixed to the second fixing portion 7022, and the other end is detachably fixed to the first fixing portion 7024. An end of the filling pad 7044 is detachably fixed to the second fixing portion 7022, and the other end is detachably fixed to the first fixing portion 7024. In a case where the filling pad 7042 and the filling pad 7044 need to be fixed at positions with different horizontal heights, two filling pads of different shapes may be selected, for example, reference may be made to filling pads of different shapes in FIG. 10A.

A fixing manner of the filling pad shown in FIGS. 7A and 7B is different from that in FIGS. 1A to 1C, and can be used to correspondingly correct different scoliosis curves. FIGS. 8 and 9 exemplarily show X-ray perspective views of two different scoliosis curves, for which the fixing manners of the filling pad shown in FIGS. 1A to 1C and FIGS. 7A and 7B can be used, respectively.

FIG. 8 shows an X-ray perspective view of a C-shaped scoliosis according to an embodiment of the present disclosure. As shown in FIG. 8, the spine in the figure includes a curve (also called a C-shape curve), curvature positions are T6-L1, where the position T10 is a vertex of the curve (i.e., the point with the largest curvature), and the convexity is toward the right side of the body. Correspondingly, the fixing manner of the filling pad shown in FIGS. 1A to 1C may be adopted, and the filling pad is fixed on the right side of the garment body with a center aligned with the position of the vertex of the curve. For the curve of FIG. 8, in an embodiment where the plurality of fixing positions are provided, the implementation of selecting the fixing position can refer to FIGS. 16A to 16C.

FIG. 9 shows an X-ray perspective view of an S-shaped scoliosis according to an embodiment of the present disclosure. As shown in FIG. 9, the spine in the figure includes two curves (also referred to as an S-shaped curve), where the one with a smaller curvature is called a minor curve, curvature positions are T6-T10, the position T8 is a vertex of the minor curve, the convexity is toward the right side of the body, and the Cobb angle is 38.1°; the one with a larger curvature is called a major curve, curvature positions are T11-L3, the position L1 is the vertex of the major curve, the convexity is toward the left side of the body, and the Cobb angle is 38.6°. The fixing manner of the filling pad of FIGS. 7A and 7B can be adopted, and one filling pad is fixed to the left side of the garment body, and the other filling pad is fixed to the right side of the garment body, and centers of the two filling pads are respectively aligned with positions of the vertices of the major curve and the minor curve. For the curve situation of FIG. 9, in an embodiment in which a plurality of fixing positions are provided, the implementation of selecting the fixing position can refer to FIGS. 18A to 18C.

An example in which the filling pad is fixed to the fixing portion through the pocket is given below for illustration. For example, at least two pockets of different shapes can be designed, and when it is necessary to fix the pockets at different positions of the fixing portion, the corresponding pockets of appropriate shapes are selected. Taking the case in which the plurality of fixing positions are designed in FIG. 3 as an example, the plurality of first fixing positions may be designed to be arranged along the longitudinal direction of the garment body, so that when the pockets of corresponding shapes are fixed to the corresponding number of first fixing positions at the corresponding positions through the at least one second fixing position, the corrective pressure is applied to the user wearing the correction garment. FIGS. 10A to 10D exemplarily show three filling pads of different shapes and corresponding pockets and positions of corresponding first fixing positions, where a fixing manner of the first fixing position and the second fixing position is explained by taking the embodiment of FIG. 4 as an example, the embodiment of FIG. 5 or other fixing manners can also be applied, which is not limited by the present disclosure.

FIG. 10A shows a schematic diagram of three filling pads of different shapes according to an embodiment of the present disclosure. As shown in FIG. 10A, 1002a, 1004a and 1006a are three filling pads of different shapes, and 1030 is a sensor disposed thereon for measuring the corrective force applied by the filling pad.

FIG. 10B shows a schematic diagram of corresponding three pockets of different shapes from an inner side perspective according to FIG. 10A. The pockets 1002b, 1004b and 1006b in FIG. 10B correspond to the filling pads 1002a, 1004a and 1006a in sequence, and for the fixing manners of the pockets 1002b, 1004b and 1006b, reference may be made to FIGS. 4 and 6.

FIG. 10C shows a schematic diagram of three filling pads of different shapes placed in corresponding pockets according to FIGS. 10A and 10B. As shown in FIG. 10C, the filling pads 1002a, 1004a and 1006a are respectively placed in the pockets 1002b, 1004b and 1006b and then fixed to the fixing portion of the garment body. For the fixing manner, reference may be made to FIG. 4 and FIG. 6.

FIG. 10D shows a schematic diagram of fixation of three pockets of different shapes in corresponding first fixing positions according to FIG. 10B. Taking the first fixing portion in FIG. 10D including 7 first fixing positions as an example, the pocket 1002b may be fixed at the 1st to 4th first fixing positions from the top, the pocket 1004b may be fixed at the 2nd to 5th first fixing positions from the top, and the pocket 1006b may be fixed at the 4th to 6th first fixing positions from the top. When a pocket which is to be fixed at a higher fixing position is designed, a larger notch and a larger notch radian can be reserved for the armpit area. For example, the notch and the notch radian of the pocket 1002b are larger than the notch and the notch radian of the pocket 1004b. The pocket 1006b may be in a rectangle form and is used to fix at the waist position.

According to the filling pads of various shapes or pockets with built-in filling pads provided in the embodiments of the present disclosure, by fixing the filling pads or pockets of different shapes at first fixing positions at different positions, it is possible to provide patients with Adolescent Idiopathic Scoliosis (AIS) of different types (for example, the “C” curve and the “S” curve included in FIGS. 8 and 9), with a more comfortable scoliosis correction garment which the patients can adjust the pressure application position by themselves, thereby effectively improving the compliance of the user.

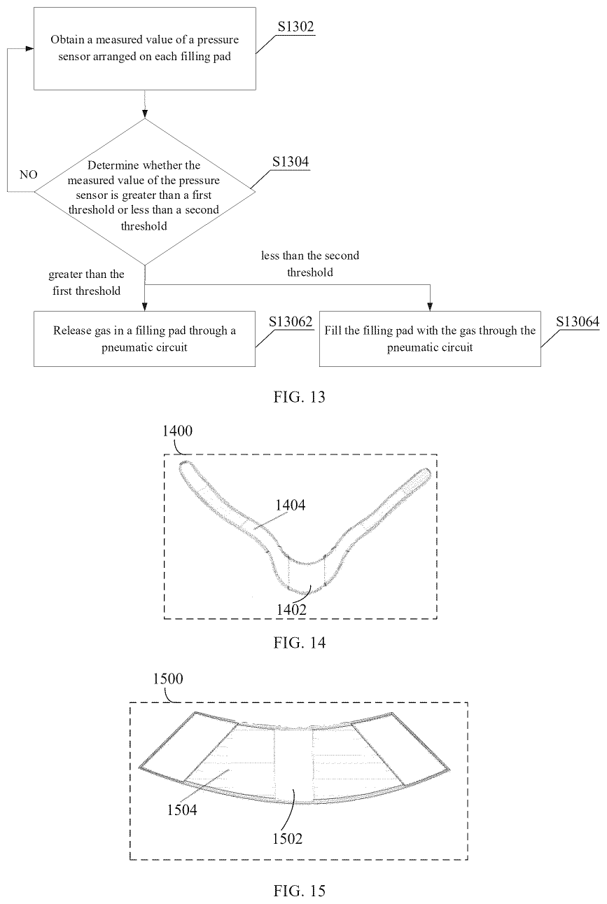

FIG. 11A shows a schematic diagram of a pneumatic circuit for controlling gas filling in a filling pad according to an embodiment of the present disclosure. The pneumatic circuit shown in FIG. 11A may include an inflation line 11012, a supply airbag 1102, and an inflation valve 11026. A first end of the inflation line 11012 is connected to the filling pad 104, and a second end of the inflation line 11012 is connected to the supply airbag 1102. An initial pressure of the supply airbag 1102 is higher than a first threshold (for the setting principle of the threshold, reference may be made to FIG. 13). The inflation valve 11026 is arranged on the inflation line 11012, and the gas flowing therein may be, for example, air. The control system 1110 can control the inflation valve 11026 to open when the measured value of the pressure sensor 1030 (e.g., the arrangement position in FIG. 10A) arranged on the filling pad 104 is less than a second threshold (for the setting principle of the threshold, reference may be made to FIG. 13).

According to the pneumatic circuit provided in the embodiments of the present disclosure, the corrective pressure applied to the wearer is automatically adjusted and enhanced, so that the curvature can be corrected by pushing the convex part of the spine while the patient is sleeping, and the pressure can be adjusted according to the sleeping posture, thereby effectively correcting the spinal deformity.

In some embodiments, the pneumatic circuit may further include an electrical pump 11024 for pre-pressurizing the system, for example, a micro pump which is small and light in weight may be used, which can generate a maximum pressure of 46 kPa to 80 kPa, to fill the filling pad 104 to a sufficient pressure, for example, less than 20 kPa, which is very convenient for the user. When the wearer uses the garment at night, the filling pad 104 may be replenished with gas only through the supply airbag 1102, thereby eliminating the noise from the electrical pump during sleep, so that the sufficient pressure can be provided to ensure the corrective support while working quietly throughout the night and allowing the wearer to sleep comfortably.

In some embodiments, the pneumatic circuit may further include an exhaust line 11014, an exhaust valve 11044 and a reserve airbag 1104. A first end of the exhaust line 11014 is connected to the filling pad 104, a second end of the exhaust line 11014 is connected to the reserve airbag 1104, and an initial pressure of the reserve airbag 1104 is the same as the initial pressure of the filling pad 104. The exhaust valve 11044 is disposed on the exhaust line 11014, so that when the measured value of the pressure sensor 1030 is greater than the first threshold, the exhaust valve 11044 is controlled to open. When the wearer uses the garment at night, the gas can be discharged into the reserve airbag 1104 when it is monitored that the pressure in the filling pad 104 is too high, thereby avoiding excessive corrective force from being applied to cause overcorrection. The reserve airbag 1104 may be designed to be larger than the filling pad 104 (e.g., referring to FIG. 11B). In the initial state, the filling pad 104 and the reserve airbag 1104 are filled with gas with the same pressure. When the gas in the filling pad 104 increases to a pressure that needs to be released, it is released into the reserve airbag 1104, and when the volume of the reserve airbag 1104 is much larger than that of the filling pad 104, the gas released from the filling pad 104 has little effect on the pressure of the reserve airbag 1104.

In some embodiments, the reserve airbag 1104 may also be used to fill the gas therein into the filling pad 104 and/or the supply airbag 1102. For example, when the filling pad 104 needs to be replenished with air, the reserve airbag 1104 can return the air to the filling pad 104, and the filling pad 104 can be reloaded in conjunction with the use of supply airbag 1102. With this special pneumatic structure, air efficiency can be increased, and the energy required for compressing the gas can be stored and reused in the entire system.

In some embodiments, the reserve airbag and the supply airbag may also be provided with safety valves to control a pressure of the reserve airbag and a pressure of the supply airbag not to be higher than respective safety values. Referring to FIG. 11A, the reserve airbag 1104 may be provided with a safety valve 11042, and the supply airbag 1102 may be provided with a safety valve 11022. The safety valve 11042 and the safety valve 11022 may both be provided with respective preset safety values. Once the pressure in the corresponding airbag exceeds the preset safety value, the safety valve will open and release the air in the corresponding airbag until the pressure drops back to the safety value to avoid over pressurization.

In some embodiments, a solenoid valve 1108 may be provided in the pneumatic circuit for each filling pad 104. By connecting the filling pad 104 to the pressure sensor 1030, a microcontroller in the control system 1110 may obtain a real-time pressure of the filling pad 104. When the real-time pressure of the filling pad 104 is greater than the first threshold or less than the second threshold, the microcontroller may control the corresponding exhaust valve 11044 or inflation valve 1102 to open, and control the solenoid valve 1108 corresponding to the filling pad 104 to open, so as to realize accurate and quiet redistribution of the pressure between the corresponding airbag and the filling pad 104 at night.

In some embodiments, the large contact area between the filling pad 104 and the body allows the pressure sensor 1030 to also be used to measure the wearer's breathing rhythm.

FIG. 11B shows a schematic diagram of an example physical connection of the pneumatic circuit diagram in FIG. 11A. Referring to FIG. 11A, the reserve airbag 1104 may be designed to be relatively large, and the electrical pump 11024 is connected to the supply airbag 1102. The reserve airbag 1104 and the supply airbag 1102 are respectively connected to the filling pad 104 (not shown in FIG. 11B) through gas lines (dashed lines in the figure). The pressure sensor 1030 may be connected to the control system 1110 by wire or wirelessly to transmit the measured pressure value in the form of a signal. The control system 1110 can be connected by wire (not shown in FIG. 11B) to each valve and the electrical pump 11024 to control the opening and closing of each valve, and control the inflation duration of the electrical pump 11024.

In other embodiments, the pneumatic circuit may not be provided with the reserve airbag, and when the pressure of the filling pad is too high and needs to be released, it can be directly discharged into the atmosphere.

FIG. 12 shows a schematic diagram of another pneumatic circuit according to an embodiment of the present disclosure. The pneumatic circuit shown in FIG. 12 differs from FIG. 11A only in that the reserve airbag is not provided in FIG. 12, and the second end of the exhaust line 1201 provided with the exhaust valve 1202 is directly connected to the atmosphere, the initial pressure of the filling pad 104 is equal to the atmospheric pressure, and the exhaust valve is controlled to be provided on the exhaust line, so that when the pressure of the corresponding filling pad 104 measured by the pressure sensor 1030 is greater than the first threshold, the control system 1110 controls the exhaust valve 1202 to open and discharge the gas in the filling pad 104 into the atmosphere.

FIG. 13 shows a flowchart schematic diagram of controlling gas in a filling pad through a pneumatic circuit according to an embodiment of the present disclosure. The embodiments of the present disclosure may include the following steps S1302 to S13066.

In the step S1302, a measured value of a pressure sensor arranged on each filling pad is obtained.

In some embodiments, the control system may obtain the measured values of the individual pressure sensors at a certain frequency, such as every minute, every 5 minutes, every 10 minutes, every hour, etc.

In the step S1304, it is determined whether the measured value of the pressure sensor is greater than a first threshold or less than a second threshold.

In some embodiments, a standard value of the pressure may be preset for the filling pad. For example, the standard value may be set according to the fixing position corresponding to the shape of the filling pad, and/or may be set according to the spinal curvature of the wearer. The first threshold may be the standard value plus a first preset value, and the second threshold may be the standard value minus a second preset value, and the first preset value and the second preset value may be the same or different.

In the step S13062, when the measured value of the pressure sensor is greater than the first threshold, the gas in the filling pad is released through the pneumatic circuit.

In the step S13064, when the measured value of the pressure sensor is less than the second threshold, the filling pad is filled with the gas through the pneumatic circuit.

If the measured value of the pressure sensor is neither greater than the first threshold nor less than the second threshold, the step S1302 is returned to.

The scoliosis correction garment provided by the embodiments of the present disclosure may also include a shoulder fixing belt and/or a pelvic fixing belt, which is used to provide the corrective force to the shoulder and/or the pelvis when necessary.

FIG. 14 shows a schematic diagram of a shoulder fixing belt according to an embodiment of the present disclosure. As shown in FIG. 14, the shoulder fixing belt 1400 may include a central portion 1402 and two belt-like portions 1404 extending from the central portion to both ends, and the central portion 1402 and the two belt-like portions 1404 may be integrally designed. The central portion 1402 is adjustably fixed to the fixing portion, and the two belt-like portions 1404 are respectively wrapped around a shoulder at a side and then their ends are fixed to the fixing portion.

In some embodiments, the central portion 1402 may be wider than the belt-like portion 1404. For example, the central portion 1402 may be fixed to the fixing portion in the middle of the back of the garment body through the magic tape or Velcro, and the two belt-like portions 1404 may respectively wrap around a shoulder at a side and then cross in the back and wrap around the body to the fixing portion in the middle of the front of the garment body, and then the two ends may be connected together by the magic tape (referring to FIGS. 16A to 19).

In some embodiments, the central portion 1402 may include an air cotton pad, and the belt-like portion 1404 may be a woven elastic band. The air cotton pad is sewn to the woven elastic band on both sides, and an edge of the air cotton pad is covered by a v-fold elastic band.

According to the shoulder fixing belt provided in the embodiments of the present disclosure, the center portion of the shoulder fixing belt is adjustably fixed to the fixing portion, and the two belt-like portions 1404 are respectively wrapped around a shoulder at a side and then their ends are fixed to the fixing portion, so that the wearer's shoulders can be fixed and the back can be kept straight.

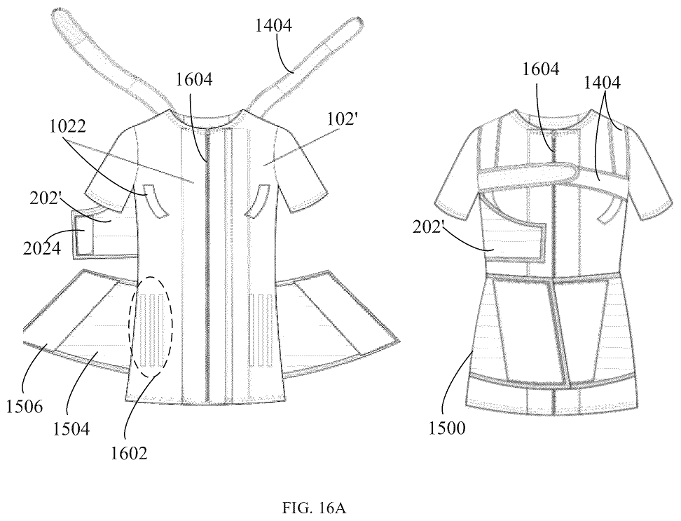

FIG. 15 shows a schematic diagram of a pelvic fixing belt according to an embodiment of the present disclosure. As shown in FIG. 15, the pelvic fixing belt 1500 can be designed in an integrated manner, a central portion 1502 can be adjustably fixed to the lower middle portion of the fixing portion, and after portions 1504 of the central portion extending to both ends wrap around the garment body, their two ends can be detachably connected. The entire pelvic fixing belt 1500 surrounds the wearer's pelvis and can apply the pressure to keep the pelvis in a proper position.

In some embodiments, the pelvic fixing belt 1500 may be formed by sewing the woven elastic band and the elastic fabric, and an edge of the pelvic fixing belt 1500 is covered by the v-fold elastic band.

In some embodiments, the pelvic fixing belt 1500 may have a hook-surface magic tape sewn on both side edges for fixing to the fixing portion sewn with the loop-surface magic tape in the middle of the front of the garment body (referring to FIGS. 16A to 19).

FIGS. 16A, 16B and 16C show schematic diagrams of a fixing position of a filling pad and a wearing manner of a shoulder fixing belt 1400 and a pelvic fixing belt 1500 for a spinal curve in FIG. 8. In FIGS. 16A, 16B and 16C, the filling pad is exemplarily placed in the pocket, and the pocket is fixed to the fixing portion of the garment body in the manner of FIGS. 4 and 6; and the garment body 102′ is exemplified as a short-sleeved bodysuit, such as a round-neck undergarment with a length covering the buttocks.

FIG. 16A shows a front schematic diagram of a wearing manner of a shoulder fixing belt and a pelvic fixing belt according to an embodiment of the present disclosure. FIG. 16B shows a back schematic diagram of a wearing manner of a shoulder fixing belt and a pelvic fixing belt according to an embodiment of the present disclosure. As shown in FIGS. 16A and 16B, a central portion 1402 of the shoulder fixing belt 1400 may be fixed to an upper portion of a first fixing portion 1024′ at the back of the garment body 102′ through the magic tape, and two belt-like portions 1404 respectively wrap around a shoulder at a side and then cross at the back and wrap around the body to an upper middle portion of a second fixing portion 1022 at the front of the garment body 102′, and then two ends of the two belt-like portions 1404 are connected together by means of the magic tape. The fixing position of the central portion 1402 of the shoulder fixing belt 1400 can be adjusted according to different body proportions of different wearers, which is convenient for different scoliosis patients to wear the garment and improve the universality of the garment.

In some embodiments, the garment body 102′ may further be provided with support bones 1602, which may be provided on both sides of the second fixing portion 1022 and the first fixing portion 1024′. The support bones 1602 may be made of a material including plastic, polyester, polyester fiber, silicone or resin, etc., to achieve a stabilizing effect.

In some embodiments, the garment body 102′ may be provided with a zipper 1604, which may be provided, for example, at the center of the front of the garment body 102′, and a length of the zipper 1604 may extend from the neckline to the hem (FIG. 16A is only an example, not limited thereto), or from the neckline to the middle. The design of the zipper 1604 is convenient for wearing and taking off, and is convenient for the user to use.

FIG. 16C shows a side schematic diagram of a wearing manner of a shoulder fixing belt and a pelvic fixing belt according to an embodiment of the present disclosure. Referring to FIGS. 16B and 16C together, for the case where the curvature positions are T6-L1 and the position T10 is the vertex of the curve shown in FIG. 8, the second fixing positions 2022′ provided with the hook and loop magic tape of the pocket 202′ with the built-in filling pad are fixed to the 2nd to 5th first fixing positions 10242′ from the top, and the third fixing position 2024 is fixed to the second fixing portion 1022, so that the pocket wraps around the right side of the body for fixation, and a direction in which the force is applied is approximately within the range of the 2nd to 3rd second fixing positions 2022′.

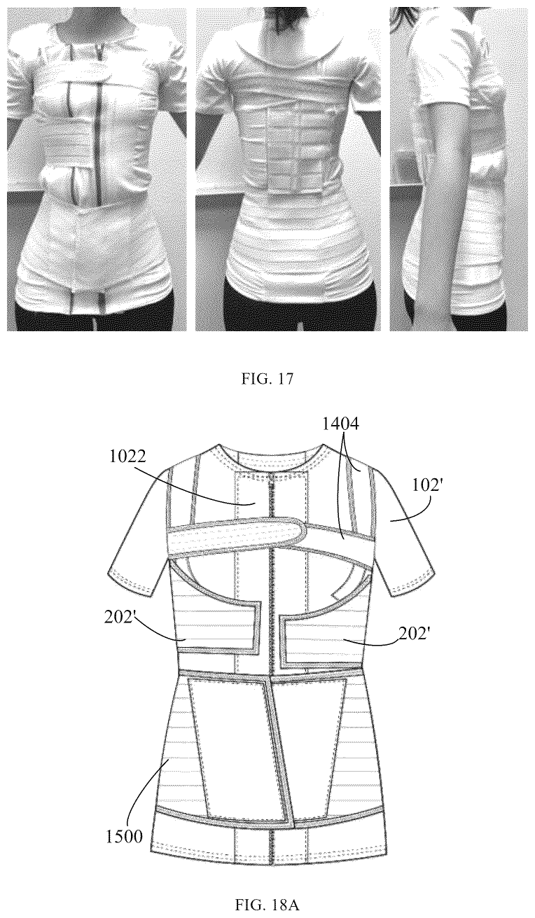

FIG. 17 shows a schematic diagram of a user actually wearing a clothing including a shoulder fixing belt and a pelvic fixing belt according to FIGS. 16A to 16C. The wearing manner of the shoulder fixing belt and the pelvic fixing belt and the fixing manner and the fixing position of the pocket in FIG. 17 are the same as those in FIGS. 16A, 16B and 16C.

FIGS. 18A, 18B and 18C show schematic diagrams of a fixing position of a filling pad and another wearing manner of a shoulder fixing belt 1400 and a pelvic fixing belt 1500 for a spinal curve in FIG. 8. In FIGS. 18A, 18B and 18C, the filling pad is exemplarily placed in the pocket, and the pocket is fixed to the fixing portion of the garment body in the manner of FIGS. 4 and 6; and the garment body 102′ is exemplified as a short-sleeved bodysuit, such as a round-neck undergarment with a length covering the buttocks.

FIG. 18A shows a front schematic diagram of another wearing manner of a shoulder fixing belt and a pelvic fixing belt according to an embodiment of the present disclosure. FIG. 18B shows a back schematic diagram of another wearing manner of a shoulder fixing belt and a pelvic fixing belt according to an embodiment of the present disclosure. As shown in FIGS. 18A and 18B, a central portion 1402 of the shoulder fixing belt 1400 may be fixed to an upper portion of a first fixing portion 1024′ at the back of the garment body 102′ through the magic tape, and two belt-like portions 1404 respectively wrap around a shoulder at a side and then cross at the back and wrap around the body to an upper middle portion of a second fixing portion 1022 at the front of the garment body 102′, and then two ends of the two belt-like portions 1404 are connected together by means of the magic tape. The fixing position of the central portion 1402 of the shoulder fixing belt 1400 can be adjusted according to different body proportions of different wearers, which is convenient for different scoliosis patients to wear the garment and improve the universality of the garment.

In some embodiments, the garment body 102′ may further be provided with support bones 106, which may be provided on both sides of the second fixing portion 1022 and the first fixing portion 1024′. The support bones 106 may be made of a material including plastic, polyester, polyester fiber, silicone or resin, etc., to achieve a stabilizing effect.

FIG. 18C shows a side schematic diagram of another wearing manner of a shoulder fixing belt and a pelvic fixing belt according to an embodiment of the present disclosure. Referring to FIGS. 18B and 18C together, for the minor curve with the curvature positions T6-T10 and the position T8 as the vertex and the major curve with the curvature positions T11-L3 and the position L1 as the vertex as shown in FIG. 9, the second fixing positions 2022′ provided with the hook and loop magic tape of the two pockets 202′ with the built-in filling pads are respectively fixed to the 1st to 4th first fixing positions 10242′ from the top (at the right), and are fixed to the 5th to 7th first fixing positions 10242′ from the top (at the left), so as to apply forces to the vertices of the two curves, respectively.

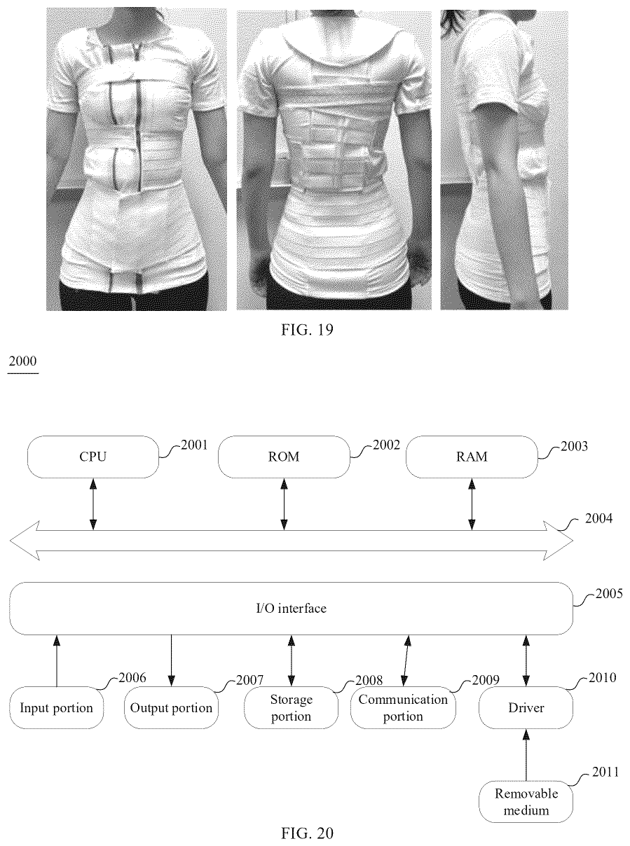

FIG. 19 shows a schematic diagram of a user actually wearing a clothing including a shoulder fixing belt and a pelvic fixing belt according to FIGS. 18A to 18C. The wearing manner of the shoulder fixing belt and the pelvic fixing belt and the fixing manner and the fixing position of the pocket in FIG. 19 are the same as those in FIGS. 18A, 18B and 18C.

The scoliosis correction garment provided by the embodiments of the present disclosure can be used as an anisotropic nighttime brace to provide the corrective pressure that can be monitored and controlled in real time to ensure that the wearer is subjected to the appropriate corrective force, which can prevent excessive or insufficient pressure from being applied to the wearer, thereby improving the effectiveness of the treatment; the corrective force is stably applied by providing the fixing portion on the garment body; the adjustable pocket design can adapt to the filling pads of adolescent idiopathic scoliosis patients with different curves, eliminating the need for tailor-made adjustments like traditional braces, thereby reducing the manufacturing cost. By designing three pocket fixing manners, the difficulties and inconveniences caused by wearing the anisotropic nighttime brace are minimized, the quality of life of the wearer is improved, and the user compliance is effectively improved.

FIG. 20 shows a schematic structural diagram of an electronic device according to an embodiment of the present disclosure. The electronic device in FIG. 20 may be an implementation of the control system in the above-mentioned pneumatic circuit. It should be noted that the device shown in FIG. 20 is only an example of a computer system, and should not bring any limitation to the functions and scope of use of the embodiments of the present disclosure.

As shown in FIG. 20, the device 2000 including a central processing unit (CPU) 2001 that may perform various appropriate actions and processes according to a program stored in a Read Only Memory (ROM) 2002 or a program loaded from a storage portion 2008 into a Random Access Memory (RAM) 2003. In the RAM 2003, various programs and data necessary for the operation of the device 2000 are also stored. The CPU 2001, ROM 2002, and RAM 2003 are connected to each other by a bus 2004. An input/output (I/O) interface 2005 is also connected to the bus 2004.

The following components are connected to the I/O interface 2005: an input portion 2006 including a keyboard, a mouse, and the like; an output portion 2007 including a display such as a Cathode Ray Tube (CRT), a Liquid Crystal Display (LCD), and the like, and a speaker; the storage portion 2008 including a hard disk and the like; and a communication portion 2009 including a network interface card such as a LAN card, a modem, or the like. The communication portion 2009 performs communication processing via a network such as the internet. A driver 2010 is also connected to the I/O interface 2005 as necessary. The removable medium 2011, such as a magnetic disk, an optical disk, a magneto-optical disk, a semiconductor memory, or the like, is mounted on the driver 2010 as necessary, so that a computer program read out therefrom is mounted into the storage portion 2008 as necessary.

In particular, according to an embodiment of the present disclosure, the process as described above with reference to the flowchart may be implemented as a computer software program. For example, embodiments of the present disclosure include a computer program product including a computer program carried on a computer-readable medium, and the computer program includes program codes for performing the method shown in the flowchart. In such an embodiment, the computer program may be downloaded and installed from the network through the communication portion 2009 and/or installed from the removable medium 2011. The computer program, when executed by the central processing unit (CPU) 2001, executes the above functions defined in the system of the present disclosure.

It should be noted that the computer-readable medium shown in the present disclosure may be a computer-readable signal medium or a computer-readable storage medium or any combination thereof. The computer-readable storage medium may be, for example, but not limited to, an electrical, magnetic, optical, electromagnetic, infrared, or semiconductor system, apparatus or equipment, or any combination thereof. Examples of the computer-readable storage medium may include, but are not limited to: an electrical connection with at least one wire, a portable computer disk, a hard disk, a random-access memory (RAM), a read-only memory (ROM), an erasable programmable read-only memory (EPROM or flash memory), an optical fiber, a portable compact disk read-only memory (CD-ROM), an optical storing equipment, a magnetic storing equipment, or any suitable combination thereof. In the present disclosure, the computer-readable storage medium may be any tangible medium that includes or stores a program, and the program may be adopted by or in combination with an instruction executing system, apparatus or equipment. In addition, in the present disclosure, the computer-readable signal medium may include a data signal propagated in a baseband or as part of a carrier wave that carries a computer-readable program code. The data signals as propagated may take a variety of forms, including but not limited to electromagnetic signals, optical signals, or any suitable combination thereof. The computer-readable signal medium may also be any computer-readable medium that sends, propagates, or transmits the program as adopted by or in combination with the instruction executing system, apparatus or equipment. The program code included on the computer-readable medium may be transmitted via any suitable medium, including but not limited to wireless, wire, fiber optic cable. RF, etc., or any suitable combination thereof.

The flowchart and block diagrams in the drawings illustrate architecture, functionality, and operation possibly implemented by systems, methods and computer program products according to various embodiments of the present disclosure. In this regard, each block in the flowchart or block diagrams may represent a module, segment, or portion of code, which includes one or more executable instructions for implementing the specified logical function. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams or flowchart and a combination of blocks in the block diagrams or flowchart may be implemented by special purpose hardware-based systems that perform the specified functions or operations, or a combination of special purpose hardware and computer instructions.

The processes involved in the embodiments of the present disclosure may be implemented by software or by hardware. The steps in the module execution process may also be set in the processor. For example, it may be described as follows: a processor includes an obtaining module, a determining module, and an adjusting module; the obtaining module may be configured to obtain a measured value of a pressure sensor provided on each filling pad; the determining module may be configured to determine whether the obtained measured value of the pressure sensor is greater than a first threshold or less than a second threshold; and the adjusting module may be configured to adjust the corresponding valve according to a determination result of the determining module. The names of these modules do not constitute a limitation on the module itself in some cases. For example, the obtaining module may also be described as a “module for obtaining the measured value of the pressure sensor”.

As another aspect, the present disclosure further provides a computer-readable medium, which may be included in the device described in the above embodiments; or may exist independently without being assembled into the device. The above computer-readable medium carries one or more programs which, when executed by a device, cause the device to implement: obtaining a measured value of a pressure sensor provided on each filling pad; determining whether the obtained measured value of the pressure sensor is greater than a first threshold or less than a second threshold; when the measured value of the pressure sensor is greater than the first threshold, releasing gas in the filling pad through a pneumatic circuit; and when the measured value of the pressure sensor is less than the second threshold, filling the filling pad with the gas through the pneumatic circuit.

Embodiments of the present disclosure provide a computer program product or a computer program, which includes computer instructions stored in a computer-readable storage medium. A processor of a computer device reads the computer instructions from the computer-readable storage medium, and the processor executes the computer instructions, so that the computer device executes the methods provided in the above-mentioned various implementations.

Example embodiments of the present disclosure are specifically shown and described above. It should be understood that the present disclosure is not limited to the detailed structures, configurations or implementations described herein; on the contrary, the present disclosure is intended to cover various modifications and equivalent configurations included in the spirit and scope of the appended claims.

What is claimed is:

1. A scoliosis correction garment, comprising:
a garment body, provided with a fixing portion; and
a filling pad, detachably fixed to the garment body through the fixing portion to apply a corrective pressure to a scoliosis position of a user, wherein the corrective pressure is adjusted by controlling gas filling in the filling pad.

2. The garment according to claim 1, further comprising: a pocket, comprising a flexible material and configured to accommodate the filling pad, and the filling pad is fixed to the fixing portion through the pocket.

3. The garment according to claim 2, wherein the fixing portion is provided with a plurality of first fixing positions, the pocket is provided with at least one second fixing position, and the at least one second fixing position is selectively fixed to a corresponding number of first fixing positions among the plurality of first fixing positions to enable the pocket to be fixed at a selectable position of the fixing portion.

4. The garment according to claim 3, wherein the plurality of first fixing positions are arranged along a longitudinal direction of the garment body, and the pocket comprises at least two pockets of different shapes to apply the corrective pressure to the user wearing the garment in a case where pockets of corresponding shapes are fixed to a corresponding number of first fixing positions at corresponding positions through the at least one second fixing position.

5. The garment according to claim 4, wherein the plurality of first fixing positions are arranged in two columns along the longitudinal direction of the garment body, and each column of first fixing positions is configured to fix the second fixing position on the same side.

6. The garment according to claim 3, wherein the fixing portion comprises a first fixing portion provided at back of the garment body, and the plurality of first fixing positions are provided on the first fixing portion.

7. The garment according to claim 6, wherein the first fixing positions comprise connection buckles fixed to the first fixing portion, and the second fixing position comprises a hook and loop magic tape, and the hook and loop magic tape is available for self-bonding by passing through a connection buckle.

8. The garment according to claim 7, wherein the first fixing positions comprise a plurality of woven elastic bands fixed to the first fixing portion, the plurality of woven elastic bands are arranged along a longitudinal direction of the garment body and are extended along a transverse direction of the garment body, the connection buckles are sewn on the first fixing portion, and the woven elastic bands pass through corresponding connection buckles.

9. The garment according to claim 6, wherein the fixing portion further comprises a second fixing portion provided at front of the garment body, the pocket is further provided with at least one third fixing position, and the at least one third fixing position is fixed to the second fixing portion; and
wherein the third fixing position comprises a hook-surface magic tape, and the second fixing portion comprises a loop-surface magic tape.

10. The garment according to claim 2, wherein the pocket is formed by sewing a woven elastic band and an elastic and light-transmitting fabric; and
wherein an edge of the pocket is covered by a v-fold elastic band.

11. The garment according to claim 1, wherein the corrective pressure is adjusted by controlling the gas filling in the filling pad through a pneumatic circuit,
wherein the pneumatic circuit comprises a pressure sensor provided on the filling pad, and when a measured value of the pressure sensor is greater than a first threshold, gas in the filling pad is released through the pneumatic circuit, and
when the measured value of the pressure sensor is less than a second threshold, the filling pad is filled with the gas through the pneumatic circuit; and
wherein the pneumatic circuit comprises an inflation line, a supply airbag and an inflation valve, a first end of the inflation line is connected to the filling pad, a second end of the inflation line is connected to the supply airbag, an initial pressure of the supply airbag is higher than the first threshold, and the inflation valve is provided on the inflation line to control the inflation valve to open when the measured value of the pressure sensor is less than the second threshold.

12. The garment according to claim 11, wherein the pneumatic circuit comprises an exhaust line, an exhaust valve and a reserve airbag, a first end of the exhaust line is connected to the filling pad, a second end of the exhaust line is connected to the reserve airbag, an initial pressure of the reserve airbag is the same as an initial pressure of the filling pad, and the exhaust valve is provided on the exhaust line to control the exhaust valve to open when the measured value of the pressure sensor is greater than the first threshold.

13. The garment according to claim 12, wherein the reserve airbag is further configured to fill the filling pad and/or the supply airbag with gas therein; and
wherein the reserve airbag and the supply airbag are both provided with safety valves to control a pressure of the reserve airbag and a pressure of the supply airbag to be not higher than respective safety values.

14. The garment according to claim 11, wherein the pneumatic circuit comprises an exhaust line and an exhaust valve, a first end of the exhaust line is connected to the filling pad, a second end of the exhaust line is connected to atmosphere, an initial pressure of the filling pad is equal to an atmospheric pressure, and the exhaust valve is provided on the exhaust line to control the exhaust valve to open when the measured value of the pressure sensor is greater than the first threshold.

15. The garment according to claim 1, wherein a shape of the filling pad is designed according to a body contour of the user; and
the garment further comprises: a shoulder fixing belt, comprising a central portion and two belt-like portions extending from the central portion to both ends, the central portion is adjustably fixed to the fixing portion, and the two belt-like portions are respectively wrapped around a shoulder at a side and then ends of the two belt-like portions are fixed to the fixing portion.

16. The garment according to claim 15, wherein the fixing portion comprises a first fixing portion provided at back of the garment body and a second fixing portion provided at front of the garment body, the central portion is adjustably fixed to the first fixing portion, and the two belt-like portions are respectively wrapped around the shoulder at the side and then the ends of the two belt-like portions are fixed to the second fixing portion.

17. The garment according to claim 15, wherein the central portion is wider than the belt-like portions.

18. The garment according to claim 15, wherein the central portion comprises an air cotton pad, the belt-like portions are woven elastic bands, the air cotton pad is sewn to the woven elastic bands on both sides, and an edge of the air cotton pad is covered by a v-fold elastic band.

19. The garment according to claim 1, further comprising: a pelvic fixing belt, a central portion of which is adjustably fixed to a lower middle portion of the fixing portion, and portions of the central portion of the pelvic fixing belt extending to both ends are wrapped around the garment body and then two ends of the portions are detachably connected; and
wherein the pelvic fixing belt is formed by sewing a woven elastic band and an elastic fabric, and an edge of the pelvic fixing belt is covered by a v-fold elastic band.

20. The garment according to claim 1, further comprising support bones provided on both sides of the fixing portion;
wherein the garment body is made of at least one of: 100% cotton, cotton blend, or synthetic fiber blend; and
wherein the garment body is a short-sleeved top or a short-sleeved bodysuit.
